(19) United States
(12) Patent Application Publication
(10) Pub. No.: US 2020/0291483 A1
(43) Pub. Date: Sep. 17, 2020

(54) NOVEL WORKFLOW FOR EPIGENETIC-BASED DIAGNOSTICS OF CANCER

(71) Applicant: The Regents of the University of California, Oakland, CA (US)

(72) Inventors: Varun Govil, La Jolla, CA (US); Zhijian Li, La Jolla, CA (US); Ruiyuan Zhang, La Jolla, CA (US); Ishan Goyal, La Jolla, CA (US)

(21) Appl. No.: 16/794,065

(22) Filed: Feb. 18, 2020

Related U.S. Application Data
(60) Provisional application No. 62/807,695, filed on Feb. 19, 2019.

(51) Int. Cl. C12Q 1/6886; C12Q 1/28; C12Q 1/34

(52) U.S. Cl. CPC C12Q 1/6886; C12Q 2600/154; C12Q 1/34; C12Q 1/28

(57) ABSTRACT

Systems and methods are provided for diagnosing cancer by using promoter methylation as an indicator of interest. Key promoter regions of interest are first identified via supervised or unsupervised machine learning applied to the Cancer Genome Atlas via a silico predictive tool. After this, a specially-designed assay is used to detect the presence of these hyper-methylated regions of interest and provide a quantitative, fluorescent readout in order to generate clinical insight. In addition, special advances in material science and microfluidics are used to enhance the sensitivity and specificity of the assay. The workflow is then completed via integration into a smartphone application that provides the necessary data and helps streamline doctor-patient communication.

### CROSS-REFERENCE TO RELATED APPLICATION

This application claims priority under 35 U.S.C. § 119(e) to U.S. Provisional Application Ser. No. 62/807,695 filed Feb. 19, 2019, the contents of which are each incorporated by reference into the present disclosure.

### SEQUENCE LISTING

The instant application contains a Sequence Listing which has been filed electronically in ASCII format and is hereby incorporated by reference in its entirety. Said ASCII copy, created on May 4, 2020, is named 114198-0501_SL.txt and is 5,148 bytes in size.

### FIELD OF USE

The present disclosure is directed to systems and methods for performing a liquid biopsy.

### BACKGROUND

Currently, there are several techniques used for the detection of specific cancers, including imaging techniques such as computed tomography (CT), mammography for breast cancer diagnoses, or positron emission tomography (PET) scans. Although imaging techniques are often capable of detecting cancer with high accuracy, they suffer from being unable to distinguish benign from malignant tumors.

The gold standard for cancer diagnosis is tissue histopathology, but often has significant drawbacks. For example, techniques such as fine needle aspiration (FNA) and core biopsy are highly invasive and are required for the extraction of suspected tumor tissue and subsequent histological evaluation. The method has also proven to be ineffective. First, comprehensive characterization of multiple tumor specimens obtained from the same patient illustrated spatial heterogeneity as well as recurrences between the primary and local tumor in the same patient. This heterogeneity poses a pivotal challenge to guide clinical decision-making in oncology, as biopsies may be inaccurate in capturing the complete tumor genome for an individual patient. In addition, a significant barrier to biomarker testing is the availability of an adequate amount of tissue due to increasing diagnostic demands and declining amounts of tissue per patient. Finally, tissue biopsies also increase the cost of patient care and the turnaround time for getting results, thus impacting the decision-making process.

To circumvent some of the negative aspects of traditional tissue biopsies, researchers have turned to a new approach, termed liquid biopsy. This approach relies on analyzing bits of tumor material that are found in blood, urine, and saliva. Scientists have unearthed a mechanism that demonstrates how tumors shed some DNA out of the cell, which then begins to circulate throughout the bloodstream and can be more easily analyzed than traditional tissue samples. In recent years, much attention has been focused on utilizing this circulating tumor DNA (termed ctDNA) as a biomarker of disease status. This ctDNA is essentially short nucleic fragments (less than 200 bp) found in plasma after being released through cell apoptosis. Scientists believe that ctDNA carries the same genetic information that a traditional tissue biopsy would provide, but has the inherent advantage that the information is attained in a minimally invasive manner. In addition, it avoids the issue of tumor heterogeneity because it is able to provide a snapshot of the entire tumor genome.

Cancer patients are known to show abnormally high levels of cell-free DNA in the plasma which is typically derived from cancer cells that have undergone apoptosis or programmed cell death. Cancer cells often exhibit hypermethylation in CpG islands of certain tumor-related genes such as the promoter regions of tumor suppressor gene; these hypermethylated DNA fragments are released into the bloodstream when the cancer cells undergo apoptosis. Lee et al. (2016) Nucleic Acids Res. 44:1105-1117. Therefore, DNA methylation can be used to diagnose cancer in patients. The optimal means to characterize a tumor through the methylation profile of the plasma DNA is to derive a set of genes that are all incurring high levels of hypermethylation. Various degrees of methylation within a gene's CpG islands leads to associated levels of gene silencing, and in cancer, promoter hypermethylation has been linked to the silencing of tumor suppressor genes and tumorigenesis. Screening for gene mutations is a common strategy, but this does not reflect the current status or activity of the disease. Additionally, promoter methylation is often easier to evaluate due to its defined location within the promoter region of specific genes. Although hypermethylation seems to be a viable alternative to current cancer detection strategies, traditional methylation analyses have their own problems. Sodium bisulfite conversion is the most widely used method to distinguish unmethylated cytosines from methylated cytosines and can be coupled with various downstream detection technologies including next-generation sequencing (NGS) and PCR-based assays. Sodium bisulfite rapidly reaminates unmethylated cytosines to uracils, whereas methylated cytosines are only slowly converted. However, bisulfite treatment can induce random DNA breaks, resulting in short single-stranded DNA fragments, especially for circulating free DNA that is sparse and highly fragmented. Bisulfite treatment also leads to a reduction in sequence complexity and cannot distinguish from other methylated bases, both resulting in compromising efficiency.

### SUMMARY

The present disclosure overcomes the drawbacks of previously-known systems and methods by providing method for performing a liquid biopsy. The method includes contacting DNA probes with a sample of target ctDNA, the DNA probes having a graphene oxide (GO) interacting region and a target recognition region complementary to a target region of the target ctDNA based on biomarkers for identifying key CpG sites of the target ctDNA identified via a machine learning algorithm. For example, the machine learning algorithm generates a model that uses clustering and logistic regression based on a training data set, and corroborates the model against a validation dataset to determine its diagnostic accuracy. The machine learning algorithm may include a Random Forest algorithm and/or a LASSO regression algorithm. In addition, the DNA probes may include a fluorescent dye, and may have 10-150 base pairs. Further, the GO interacting region of the one or more DNA probes may include a high Guanine-Cytosine content.

The method further includes contacting the sample of target ctDNA with a labeled methyl-binding domain protein (MBD), determining ctDNA concentration and corresponding methylation levels of the target ctDNA based on fluorescence of the sample of target ctDNA, monitoring the determined ctDNA concentration and corresponding methylation levels of the target ctDNA on a digital health platform. The MBD label may include horse radish peroxidase (HRP) or green fluorescent protein (GFP). The method further may include pre-incubating the DNA probes with GO. In addition, the method may include contacting the sample of target ctDNA with an Exonuclease III solution and/or contacting the sample of target ctDNA with a hydrogen peroxide and 4-hydroxyphenylacetic acid solution.

In accordance with one aspect of the present invention, monitoring the determined ctDNA concentration and corresponding methylation levels of the target ctDNA on a digital health platform is used to determine whether an individual is predisposed to at least one of carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. In addition, the method may include assessing post-therapeutic effects of a medication by comparing the data indicative of the determined ctDNA concentration and corresponding methylation levels before and after a treatment using the medication.

In accordance with another aspect of the present invention, a system for performing a liquid biopsy is provided. The system includes non-transitory computer readable media having instructions that, when executed by a processor cause the processor to execute a machine learning algorithm to identify one or more biomarkers for identifying key CpG sites of a target ctDNA; a diagnostic cell-free protein-based system for determining ctDNA concentration and corresponding methylation levels of the target ctDNA based on measured fluorescence of a sample of target ctDNA in contact with DNA probes comprising a graphene oxide (GO) interacting region and a target recognition region complementary to a target region of the target ctDNA based on the identified biomarkers; and a digital health platform for monitoring the determined ctDNA concentration and corresponding methylation levels of the target ctDNA.

### BRIEF DESCRIPTION OF THE DRAWINGS

FIG. 1 illustrates an exemplary system for performing a liquid biopsy in accordance with the principles of the present invention.

FIG. 2 is a flow chart of steps of an exemplary method for performing a liquid biopsy in accordance with the principles of the present invention.

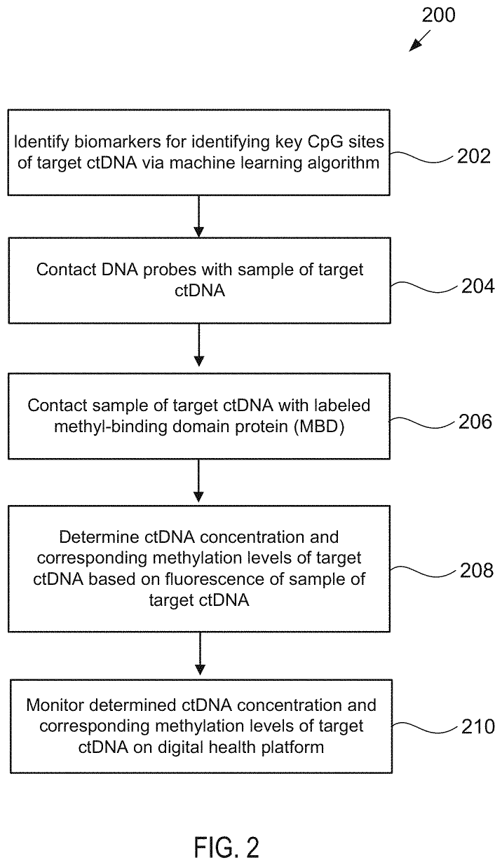

FIG. 3 illustrates a Receiving Operator Characteristic (ROC) curve used to study the output of a classifier for a binary classification problem.

FIG. 4 illustrates a comparison of the top 20 features produced from an Alpha=0.01 run with the results from Random Forest in an exemplary study conducted in accordance with the principles of the present invention.

FIG. 5 is a TGX stain-free gel of hMBD-eGFP with Unstained Protein Ladder.

FIG. 6 shows anti-His labelled with HRP of hMBD-eGFP.

FIG. 7 shows retardation bands were present in both negative controls and experimental condition (lane 3).

FIG. 8 shows retardation bands were present only symmetrically methylated DNA probes (lane 3), suggesting its specificity.

FIG. 9 shows ExoIII effect on fluorescence recovery.

FIG. 10 illustrates anti-His labelled with HRP of hmHRP Ni elution samples.

FIG. 11 shows size Exclusion Chromatography of hmHRP Ni elution 2. The blue line is the absorbance at 280 nm. Fraction 3, 4, and 5 were later loaded onto an SDS-PAGE gel with the load.

FIG. 12 illustrates anti-His labelled with HRP of hmHRP gel filtration load and fraction 3, 4, 5.

FIG. 13 is an Hm-HRP was transferred and visualized with anti-His antibody. Hm-HRP/DNA complex was confirmed by the presence of protein signal at lane 3.

FIG. 14 shows the sequences of basic constructs: mMBD-eGFP and hMBD-eGFP. FIG. 14 discloses SEQ ID NOS 7, 8, and 9, respectively, in order of appearance.

### DETAILED DESCRIPTION

In order to address key bottlenecks in liquid biopsy and noninvasive cancer detection techniques, the present invention focuses on using epigenetic determinants for diagnostic purposes. Presented here is a novel workflow for diagnosing cancer by using promoter methylation as an indicator of interest. Key promoter regions of interest are first identified via supervised or unsupervised machine learning applied to the Cancer Genome Atlas via a silico predictive tool. An algorithm that uses ground truth, or prior knowledge of what the output values should be (in this case, the disease state of the patient), is known as supervised machine learning. When the ground truth is unknown, this becomes a method known as unsupervised machine learning.

After this, a specially-designed assay is used to detect the presence of these hyper-methylated regions of interest and provide a quantitative, fluorescent readout in order to generate clinical insight. In addition, special advances in material science and microfluidics are used to enhance the sensitivity and specificity of the assay. The workflow is then completed via integration into a smartphone application that provides the necessary data and helps streamline doctor-patient communication. Proof of concept was centered around hepatocellular carcinoma.

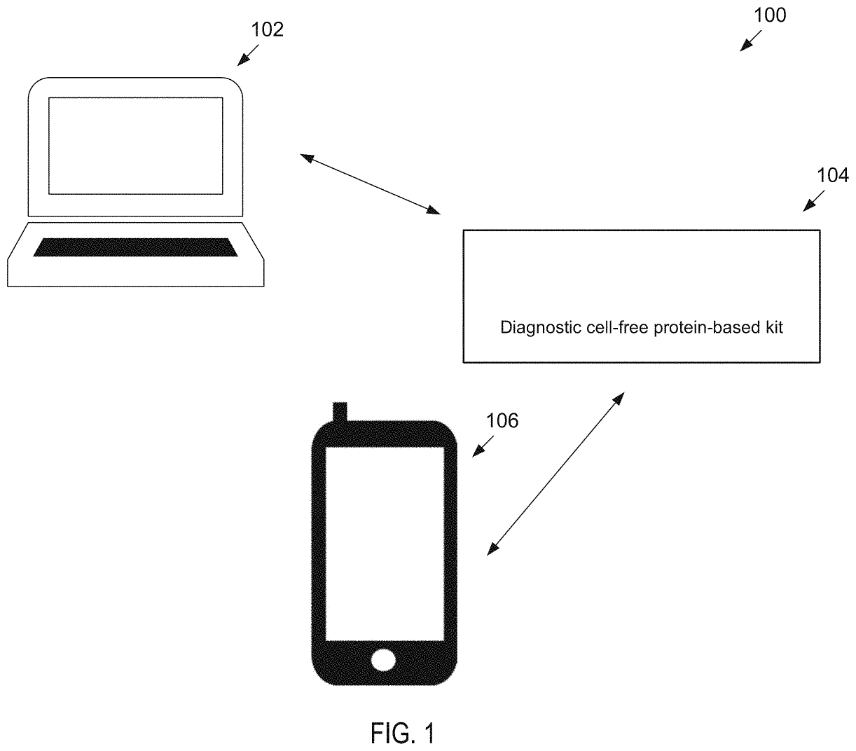

Referring now to FIG. 1, exemplary system 100 for performing a liquid biopsy in accordance with the principles of the present invention is provided. System 100 includes non-transitory computer readable media 102 having instructions that, when executed by a processor cause the processor to execute a machine learning algorithm to identify one or more biomarkers for identifying key CpG sites of a target ctDNA. For example, the machine learning algorithm may include a Random Forest algorithm or a LASSO algorithm.

The Random Forest algorithm is a supervised machine learning algorithm that can be used to solve both classification and regression problems. It is a robust algorithm that avoids over-fitting by creating decisions trees from randomly selected data samples and generating predictions from each tree. The algorithm then takes predictions from all of the trees to generate the relative feature importance for the model. It was essential to perform several data processing steps prior to running this algorithm to separate the data into feature variables (methylation markers) and target variables (HCC or healthy). The input of Random Forest includes the number of estimators, i.e., the number of decision trees in the forest, and the number of features, i.e., the total number of significant features to output from the resulting decision tree. The output of Random Forest includes Summary Statistics, i.e., error metrics, confusion matrix, and classification report, Sensitivity Analysis, e.g., AUROC curve, and Feature List, i.e., list of identified features and their respective weights. Note that the accuracy of the model improves if the number of estimators is increased, but this also introduces higher computational burden and the chance of overfitting the data.

Similar to the Random Forest procedure, LASSO is used to shrink the total number of features being used to model the data. This algorithm uses a process called regularization which penalizes features in the data in order to keep only the most important factors. The most important parameter in a LASSO regression is the alpha value. When alpha=0, a simple linear regression is being performed on the data. When alpha approaches 1 however, most of the feature coefficients reach 0 which indicates they have no weight on the model. The only downside of LASSO is that it tends to overfit the data and has a lower predictive capability when compared to Random Forest. The input of LASSO includes the alpha value, i.e., the regularization parameter, which ranges from 0-1 where alpha=0 is equivalent to a linear regression and alpha=1 causes all features to be dropped from the model. The output of LASSO includes Test Statistics, i.e., for each value of alpha, the number of features used and the test/training score for the model, and Feature List, i.e., list of identified features and their coefficients.

By running both selection algorithms, we are able to derive a subset of biomarkers that strongly contribute to the classification model. Taking an overlap of the two lists produced from each method yields a set of computationally likely disease-causing methylation loci.

For example, in a study involving the analysis of 485,000 unique CpG markers to generate a final set of CpG markers that were enhanced in HCC patients, the two analyses yielded overlapping markers where used to test sensitivity and specificity against the training and validation data sets. From the Random Forest Analysis, the number of training observations was 1752, and the number of test observations was 439. In a Random Forest prediction model, feature importances give a sense of which variables have the most effect in these models. The sklearn package has a .feature_importances_attribute that returns an array of each features importance in the model. To identify the top 30 positions that contribute to the model, the top 30 feature weights were taken and organizes in descending order in Table 1 below.

TABLE 1

| Biomarker ID | Feature Weight |
|---|---|
| X4.1324870 | 0.09226192627385134 |
| X17.80358809 | 0.059085931369960085 |
| X5.171538557 | 0.057999353985870285 |
| X4.1324877 | 0.03303274292587381 |
| X11.47624801 | 0.03174275980572196 |
| X3.101808857 | 0.02760567525917319 |
| X17.49295615 | 0.02695179769504988 |
| X10.135072960 | 0.026502243602910897 |
| X7.151106060 | 0.025569011566135204 |
| X10.103534546 | 0.017009344232785523 |
| X6.31527889 | 0.016819558850001068 |
| X10.88684020 | 0.013249692773994187 |
| X2.113931518 | 0.012937163887263686 |
| X7.151106022 | 0.011656198731186432 |
| X21.36421467 | 0.011245567212164166 |
| X10.134141823 | 0.01078311019638933 |
| X8.53851151 | 0.010115600105707685 |
| X10.14701815 | 0.00988707517481865 |
| X1.226296852 | 0.00823810513451597 |
| X17.47286802 | 0.008169863614495784 |
| X4.1324849 | 0.007778442275209609 |
| X6.41528449 | 0.007534845972666597 |
| X7.27155000 | 0.007265779808743397 |
| X11.69707285 | 0.007172146198231417 |
| X8.2879889 | 0.0067021680826400585 |
| X8.48656343 | 0.006613567990276688 |
| X18.61143902 | 0.006435928106157796 |
| X4.1324842 | 0.006322098138107917 |
| X6.16729606 | 0.006154748779256597 |
| X12.20522379 | 0.0061085520577785464 |

The confusion matrix in Tables 2-4 copied below is a summary of the prediction results generated from the model on a classification problem.

TABLE 2

| | Negative Test | Positive Test | Total |
|---|---|---|---|
| Disease Absent | True Negative (TN) | False Positive (FP) | TN + FP |
| Disease Present | False Negative (FN) | True Positive (TP) | FN + TP |
| Total | TN + FN | FP + TP | FP + TP |

TP: Case where the disease is present and the test predicts the state accurately.
FP: Case where the disease is absent, but the test fails and predicts it as present.
FN: Case where disease is present, but the test fails and predicts it as absent.
TN: Case where the disease is absent and the test predicts the state accurately.

Accuracy=((TP+TN)/Total)

Precision=(TP/(TP+FP))

Sensitivity=(TP/(TP+FN))

Specificity=(TN/(TN+FP))
F1 Score—Measure of a binary classification's accuracy by taking a weighted average of the recall and precision values.
Micro Average—Aggregates the contributions of all classes to compute the average metric, used when class imbalance is suspected. (More examples of one class than another)
Macro Average—Computes the metric independently for each class and then takes the average, thus treating all classes equally.
Weighted Average—Each class contribution to the average is weighted by the relative number of examples available for it.

TABLE 3

| | Negative Test | | Positive Test | | Total | |
|---|---|---|---|---|---|---|
| Disease | 167 | (TN) | 7 | (FP) | 174 | (TN + FP) |
| Absent | | | | | | |
| Disease | 15 | (FN) | 250 | (TP) | 265 | (FN + TP) |
| Present | | | | | | |

| | | | | | |
|---|---|---|---|---|---|
| Total | 182 | (TN + FN) | 257 | (FP + TP) | 439 |

Accuracy=(167+250/439)=0.949886

Sensitivity=(250/250+15)=0.943396

Specificity=(167/167+7)=0.959770

TABLE 4

| | | | | Support |
|---|---|---|---|---|
| | | | | (# of |
| | Precision | Recall | F1-score | Samples) |
| 0 (Healthy) | 0.92 | 0.96 | 0.94 | 174 |
| | (167/192) | (167/174) | | |
| 1 (Disease) | 0.97 | 0.94 | 0.96 | 265 |
| | (250/257) | (250/265) | | |
| micro average | 0.94 | 0.94 | 0.94 | 439 |
| macro average | 0.93 | 0.94 | 0.94 | 439 |
| weighted average | 0.94 | 0.94 | 0.94 | 439 |

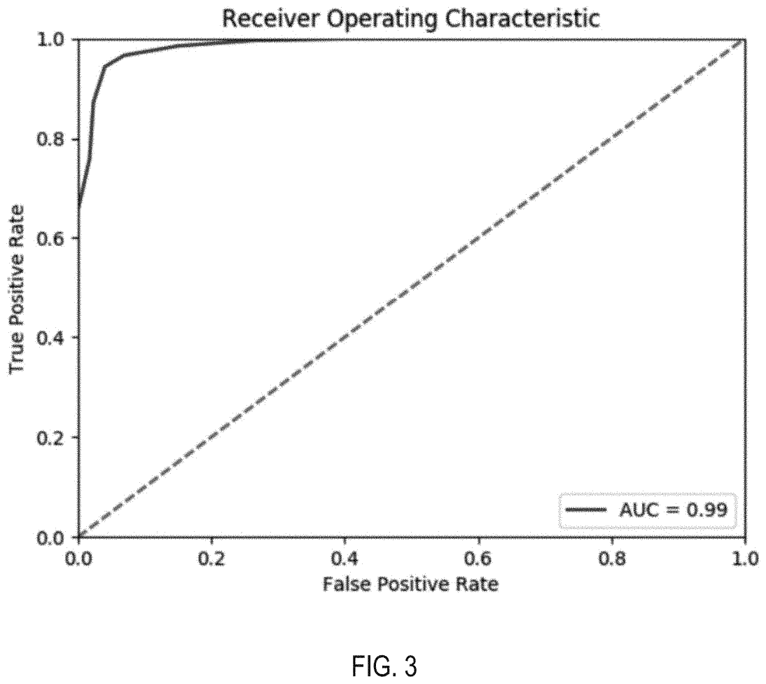

Receiving Operator Characteristic (ROC) curves are typically used to study the output of a classifier for a binary classification problem. As shown in FIG. 3, the outputs were labeled as 1 for HCC patient samples and 0 for healthy patient samples. The ROC curve maps the true positive rate (y-axis) against the false positive rate (x-axis) where the top left corner is the ideal point as this indicates a true positive rate of 1 and a false positive rate of 0. We often observe and try to optimize the area under this curve (AUC) which is some value between 0 and 1. In the ROC curve generated by this random forest model, the AUC=0.9876165690739536.

As the name suggests, the Random Forest algorithm produces a unique forest of decision trees each time it is run. As a result, each run of the algorithm produces a different set of features with unique weights. The purpose of the feature estimator is to take the output of 50 random forest algorithm instances and generate a weighted list of top features/biomarkers. Since the features included in each individual output may not be the same and may be in different orders depending upon the decision tree that was generated, it is essential to weight all these outputs. The weighted and ranked features have been listed in Table 5 copied below.

TABLE 5

| Rank | Biomarker ID |
|---|---|
| 1 | X4.1324870 (#1) |
| 2 | X4.1324849 (#21) |
| 3 | X4.1324842 (#28) |
| 4 | X11.64644496 |
| 5 | X10.88684020 (#12) |
| 6 | X11.47624801 (#5) |
| 7 | X17.80358809 (#2) |
| 8 | X4.1324877 (#4) |
| 9 | X11.1102570 |
| 10 | X7.151106060 (#9) |
| 11 | X7.151106022 (#14) |
| 12 | X21.36421467 (#15) |
| 13 | X5.171538557 (#3) |
| 14 | X6.41528449 (#22) |
| 15 | X3.101808857 (#6) |
| 16 | X7.35301161 |
| 17 | X17.49295615 (#7) |
| 18 | X7.140267061 |
| 19 | X10.6162175 |
| 20 | X12.20522379 (#30) |
| 21 | X19.14993513 |
| 22 | X10.135072960 (#8) |
| 23 | X6.170494251 |
| 24 | X22.37813041 |
| 25 | X15.55569496 |
| 26 | X1.226296852 (#19) |
| 27 | X7.139929764 |
| 28 | X8.2879889 (#25) |
| 29 | X8.124332861 |
| 30 | X10.3823804 |

A majority of the biomarkers in the features estimator are bolded indicating they match the features identified in the example random forest run. This is expected since the estimator should encompass the most likely features that will be found in any given Random Forest.

The LASSO method was run on the dataset using several different alpha parameters including alpha=[0.0001, 0.01, 0.05, 0.1, and 1]. The training score, test score, and number of features used for each alpha level are indicated in Table 6 copied below.

TABLE 6

| | | | Number of |
|---|---|---|---|
| Alpha Level | Training Score | Test Score | Features |
| Alpha = 1 | 0 | −0.004419100127760922 | 0 |
| Alpha = 0.1 | 0.15677933815289924 | 0.13977060185368662 | 2 |
| Alpha = 0.05 | 0.4381200357272692 | 0.42151384889843724 | 8 |
| Alpha = 0.01 | 0.6807028476075634 | 0.6585068207316982 | 26 |
| Alpha = 0.0001 | 0.8291845505629482 | 0.6949522662970736 | 393 |

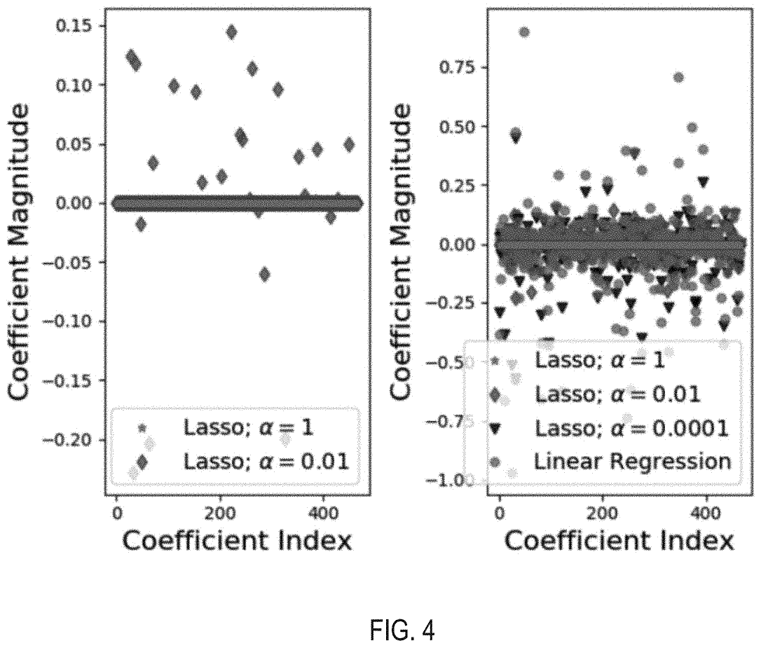

As shown in Table 6, as the value of alpha approaches 0, the number of features used in the model increases and the prediction accuracy also improves. This is accurate with how the alpha parameter works as a value of Alpha=0 is equivalent to running a linear regression and including all of the features. The visualization below is generated by the LASSO analysis and shows the various features with different coefficient magnitudes at the varying alpha levels. The spread for alpha=1 is a straight line along zero coefficient magnitude because all of the features are dropped at this alpha level. The spread for alpha=0.0001 is very similar to the linear regression spread which is the lower bound of alpha. For further analysis, we sort the top 20 features produced from the Alpha=0.01 run and compare them to the results from Random Forest as illustrated in FIG. 4.

It is important to note that as the alpha level increases, some of the features that have a significant weight at a lower level drop out of the model. After sorting based on the alpha=0.01 column, we find an overlapping 9 biomarkers (in bold) between the Random Forest and LASSO outputs as shown in Table 7 copied below.

TABLE 7

| Biomarker ID | Alpha = 0.0001 | Alpha = 0.01 | Alpha = 0.05 |
|---|---|---|---|
| X17.80358809 | 0.066168 | 0.14503 | 0 |
| X10.6162175 | 0.06678 | 0.123655 | 0 |
| X11.1102570 | 0.105655 | 0.118012 | 0.082344 |
| X21.36421467 | 0.382851 | 0.113295 | 0 |
| X1.226296852 | 0.064078 | 0.099545 | 0.002631 |
| X3.101808857 | 0.053619 | 0.096017 | 0.161699 |
| X14.23291079 | 0.088821 | 0.093794 | 0.029928 |
| X19.18874658 | 0.045012 | 0.057451 | 0.003233 |
| X19.36233435 | 0.111286 | 0.053625 | 0 |
| X8.2879889 | 0.017818 | 0.04995 | 0.014396 |
| X6.41528449 | 0.070034 | 0.045128 | 0 |
| X5.172982345 | 0.033804 | 0.039364 | 0 |
| X11.69707285 | 0.021109 | 0.033732 | 0 |
| X17.55456535 | 0.08954 | 0.022602 | 0 |
| X1.53794245 | 0.093721 | 0.017271 | 0 |
| X6.134491421 | 0.037699 | 0.005953 | 0 |
| X2.127933601 | 0.023571 | 0.003084 | 0 |
| X7.83479508 | 0.01037 | 0.002575 | 0 |
| X6.3247680 | −0.020572 | 0.000515 | 0 |
| X3.150321178 | 0 | 0 | 0 |

System 100 further includes diagnostic cell-free protein-based kit 104 for determining ctDNA concentration and corresponding methylation levels of the target ctDNA based on measured fluorescence of a sample of target ctDNA in contact with DNA probes comprising a graphene oxide (GO) interacting region and a target recognition region complementary to a target region of the target ctDNA based on the identified biomarkers. For example, kit 104 includes a microfluidic reaction platform and reagents necessary for the assay. The microfluidic reaction platform includes a reaction chamber with built-in graphene oxide coated area, a buffer reservoir, outlets, a waste bottle, a simple heating system, a Part A standard including a pre-designed ssDNA probe, and methylated DNA in different concentrations (10 pM, 1 pM, 100 fM, 50 fM, 10 fM), and a Part B standard including 100 fM pre-hybridized ctDNA with different methylation percentages (100%, 75%, 50%, 25%, 0%).

Starting material of the assay includes purified ctDNA from available commercial kits such as MagMAX™ Cell-Free DNA Isolation Kit (available from Thermo Fischer Scientific, Waltham, Mass., USA). Analysis of each methylation site requires a minimum 3 μL of undiluted ctDNA, and multiple methylation sites on the same promotor could also be characterized by this kit. It is recommended to analyze a panel of genes (8-10 methylation sites in total) for each testing. Data of gene panels may be subsequently processed by a biomarker discovery tool for the purpose of cancer diagnosis and prognosis.

The ssDNA probes should contain a 5′ methylated Cytosine on target methylation site and a 3′ end modification with fluorescent dye (e.g. FAM), which could be purchased from IDT. Regarding to the linkage type between the dye and the probe, NHS ester modification should be avoided. Preferably, target methylation site should be in proximity of 3′ end, e.g., 5 bp away. In addition, the probe should have approximately 6 bp 5′ overhang not complimentary to the target sequence.

The reagents necessary for the assay include freeze-dry MBD-HRP protein for methylation detection, HRP substrates (Hydrogen peroxide, 4-hydroxyphenylacetic acid, available from Sigma-Aldrich, St. Louis, Mo., USA), an NEB II buffer (Buffer 1), a protein-DNA binding buffer (Buffer 2), and Exonuclease III (E. coli) from NEB (Exo III).

In addition, system 100 includes digital health platform 100 in electrical communication with diagnostic cell-free protein-based system 104 for monitoring the determined ctDNA concentration and corresponding methylation levels of the target ctDNA. For example, digital health platform 100 may run on a smartphone.

In accordance with another aspect of the present invention, exemplary method 200 for performing a liquid biopsy in accordance with the principles of the present invention is provided. Method 200 may be performed using system 100 described above. For example, at step 202, one or more biomarkers for identifying key CpG sites of a target ctDNA are identified via a machine learning algorithm, e.g., Random Forest and LASSO.

At step 204, ssDNA probes comprising a graphene oxide (GO) interacting region and a target recognition region complementary to a target region of the target ctDNA based on the identified biomarkers are contacted with a sample of target ctDNA to quantify the target ctDNA. For example, the target recognition region of the DNA probes may include o_o forward ATGCACGCCGGGATTCCTCTGCTG (SEQ ID NO: 1); o_o reverse CAGCAGAGGAATCCCGGCGTGCAT (SEQ ID NO: 2); m_o forward ATGCA/iMe-dC/GCCGGGATTCCTCTGCTG (SEQ ID NO: 3); m_o reverse CAGCAGAGGAATCCCGG/iMe-dC/GTGCAT (SEQ ID NO: 4); o_m forward ATGCACGC/iMe-dC/GGGATTCCTCTGCTG (SEQ ID NO: 5); and o_m reverse CAGCAGAGGAATCC/iMe-dC/GGCGTGCAT (SEQ ID NO: 6).

In accordance with one aspect of the present invention, the probes may be pre-incubated with GO prior to contact with the sample of target ctDNA. For example, a glass slide pre-coated with graphene oxide may be inserted into the reaction chamber. The graphene oxide may be prepared in a 2 mg/mL water solution (available from Sigma-Aldrich, St. Louis, Mo., USA), and a working solution of 0.02 mg/mL may be prepared therefrom to pre-incubate the probes. In addition, Buffer 1 and Buffer 2 may be chilled on ice, and 0.5 mL of Buffer 2 may be added to dry MBD-HRP protein to make 10 nM of an MBD-HRP solution. The solution may be vortexed briefly and chilled on ice. Next, Exo III may be diluted in Buffer 1 to 50 U/mL to make an Exo III solution as each reaction will need 10 μL. The ssDNA probes are also diluted to 100 nM in Buffer 1 to make a DNA probe solution, vortexed briefly and incubated at room temperature. Additionally, purified ctDNA is diluted in Buffer 1 to make a ctDNA solution so that each reaction gets 25 μL of the solution. A water bottle is then attached to the outlet of the reaction chamber.

2 ml of Buffer 1 is added to the reaction chamber, and the outlets and the pump are turned on to allow all if the buffer to flow through the device. The outlets and the pump are then turned off. 25 μL of the DNA probe solution is spotted on corresponding wells and incubated for 20 minutes at room temperature. The outlets and the pump are turned on to dry the wells. Next, 1 ml of Buffer 1 is added to the reaction chamber, and the wash is repeated. Afterward, the outlets and pump are turned off. A fluorescent signal FO is measured with a corresponding excitation and emission wavelength. The gain should be set to optimal as seen in Table 8 copied below.

TABLE 8

| | Excitation | Emission |
|---|---|---|
| Name | (nm) | (nm) |
| FAM/fluorescein | 495 | 520 |
| MAX | 524 | 527 |
| Cy3 | 550 | 564 |
| ROX | 588 | 608 |

The heating system is then turned on to reach a temperature of 37 degrees. Next, 25 μL of the ctDNA solution is spotted to corresponding wells and incubates for at least 5 minutes. A suggested plate map is provided in Table 9 copied below.

TABLE 9

| | 1 | 2 | 3 |
|---|---|---|---|
| A | Buffer 1 | Buffer 1 | Buffer 1 |
| B | Probe from kit | Probe from kit | Probe from kit |
| C | Probe from kit + Exo III | Probe from kit + Exo III | Probe from kit + Exo III |
| D | Probe from kit + 100 pM | Probe from kit + | Probe from kit + 100 pM |
| | DNA + Exo III | 100 pM DNA + Exo III | DNA + Exo III |
| E | Probe from kit + 10 pM | Probe from kit + 10 pM | Probe from kit + 10 pM |
| | DNA + Exo III | DNA + Exo III | DNA + Exo III |
| F | Probe from kit + 1 pM | Probe from kit + 1 pM | Probe from kit + 1 pM |
| | DNA + Exo III | DNA + Exo III | DNA + Exo III |
| G | Probe from kit + 100 fM | Probe from kit + | Probe from kit + 100 fM |
| | DNA + Exo III | 100 fM DNA + Exo III | DNA + Exo III |
| H | Probe from kit + 10 fM | Probe from kit + 10 fM | Probe from kit + 10 fM |
| | DNA + Exo III | DNA + Exo III | DNA + Exo III |
| I | Probe 1 + ctDNA + Exo III | Probe 1 + ctDNA + Exo III | Probe 1 + ctDNA + Exo III |
| J | Probe 2 + ctDNA + Exo III | Probe 2 + ctDNA + Exo III | Probe 2 + ctDNA + Exo III |
| F | . . . | . . . | . . . |

10 μL of the Exo III solution is then spotted to each well, and incubated for about 90 minutes. Then fluorescent signal F1 is measured.

At step 206, the sample of target ctDNA is contacted with a labeled methyl-binding domain protein (MBD) to quantify ctDNA methylation. For example, during the 90-minute incubation, the follow steps are performed in the separate wells. A recommended plate map is shown in Table 10 copied below.

TABLE 10

| | 4 | 5 | 6 |
|---|---|---|---|
| A | Buffer 2 | Buffer 2 | Buffer 2 |
| B | Pre-hybridized | Pre-hybridized | Pre-hybridized |
| | ctDNA, 100% | ctDNA, 100% | ctDNA, 100% |
| | methylated | methylated | methylated |
| C | Pre-hybridized | Pre-hybridized | Pre-hybridized |
| | ctDNA, 75% | ctDNA, 75% | ctDNA, 75% |
| | methylated | methylated | methylated |
| D | Pre-hybridized | Pre-hybridized | Pre-hybridized |
| | ctDNA, 50% | ctDNA, 50% | ctDNA, 50% |
| | methylated | methylated | methylated |
| E | Pre-hybridized | Pre-hybridized | Pre-hybridized |
| | ctDNA, 25% | ctDNA, 25% | ctDNA, 25% |
| | methylated | methylated | methylated |
| F | Pre-hybridized | Pre-hybridized | Pre-hybridized |
| | ctDNA, 0% | ctDNA, 0% | ctDNA, 0% |
| | methylated | methylated | methylated |

Note: In row A, the ctDNA solution is replaced by Buffer 2, and subsequent procedures should be followed. The heating system is then ramped up to 70 degrees and incubated for at least 15 minutes. Then, the heating system is turned off and the device is allowed to cool below 50 degrees. Next, the outlets and pump are turned on to dry the wells.

2 ml of Buffer 2 is then added to the reaction chamber and the wash is repeated. Fluorescent signal F2 is then measured with excitation wavelength of 320 nm and emission wavelength of 410 nm. Next 50 μL of the 10 nM MBD-HRP solution is spotted to the wells, and incubated at room temperature for 20 minutes. The device is then washed with 1 ml of Buffer 2. In addition, the hydrogen peroxide is diluted to 10 nM, and the 4-hydroxyphenylacetic acid is diluted to 10 nM in Buffer 2. 20 ul of the diluted 10 nM hydrogen peroxide and 10 nM 4-hydroxyphenylacetic acid is spotted into the wells and incubates for 1 hour. Next, the fluorescent signal F3 is measured with excitation wavelength of 320 nm and emission wavelength of 410 nm.

At step 208, ctDNA concentration and corresponding methylation levels of the target ctDNA is determined based on fluorescence of the sample of target ctDNA. For example, corrected fluorescent signals may be calculated where F_{corrected}1=F1−F0, and F_{corrected}2=F3−F2, to generate external standard curves for the quantified target ctDNA and quantified methylated ctDNA, i.e., corrected fluorescence signal vs. concentration. Accordingly, the ctDNA concentration and corresponding methylation levels are determined used the two standard curves.

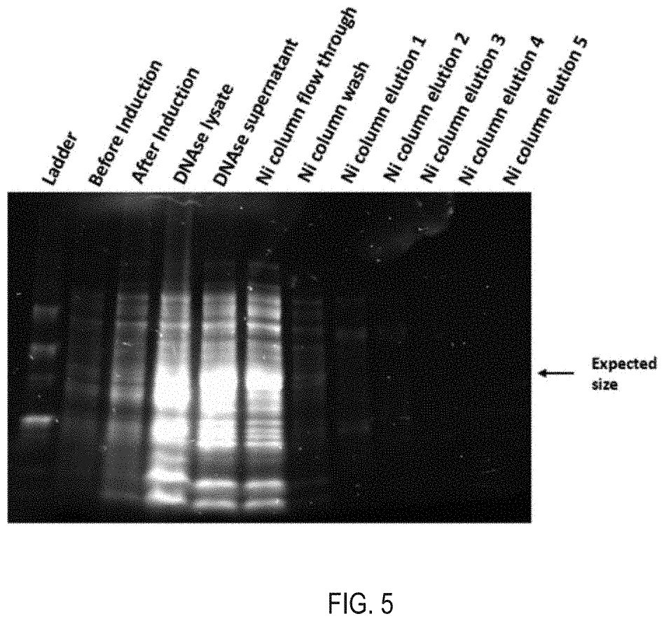

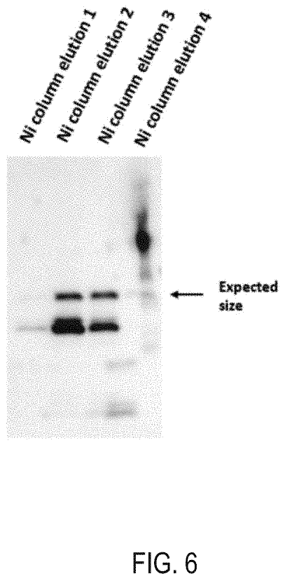

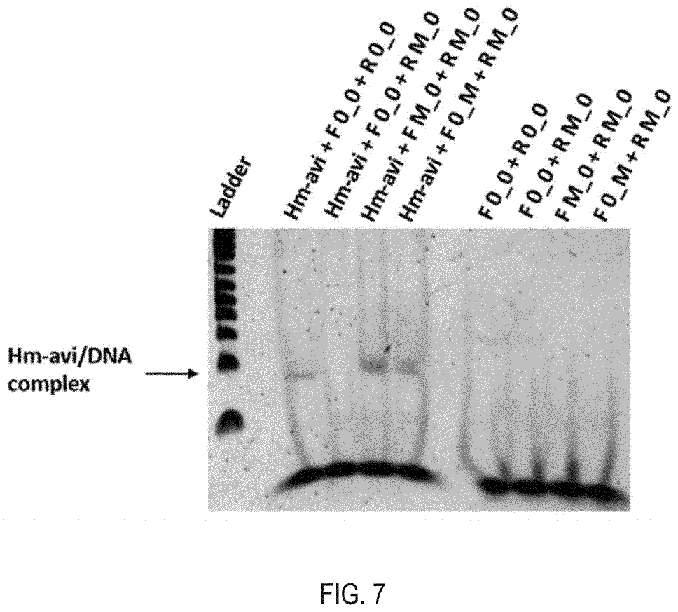

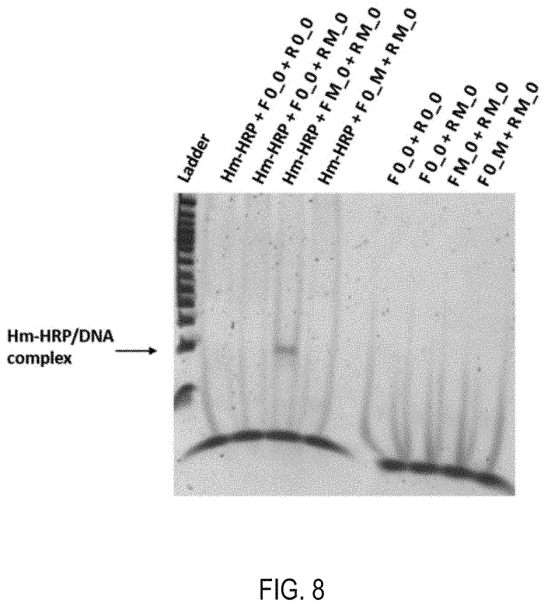

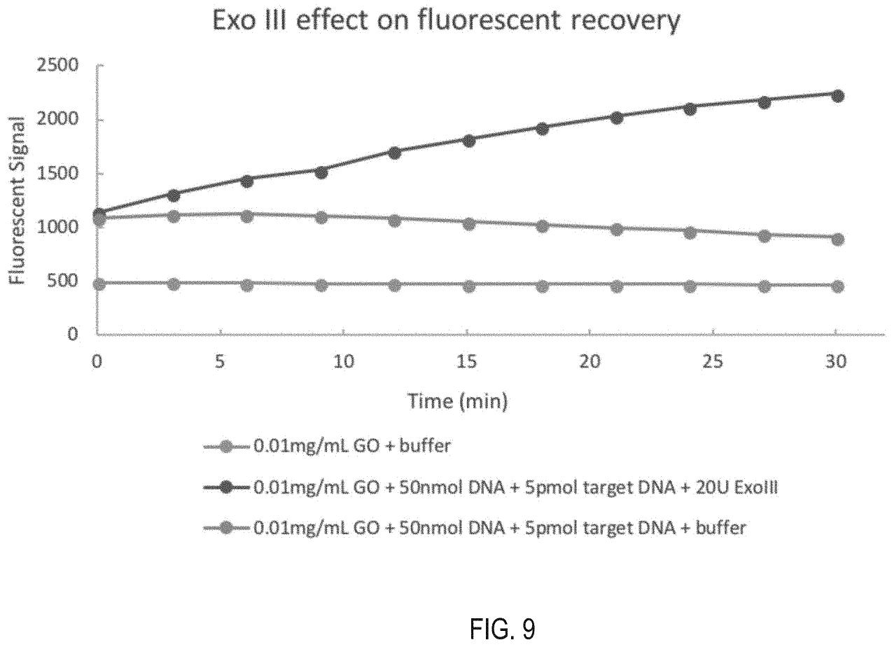

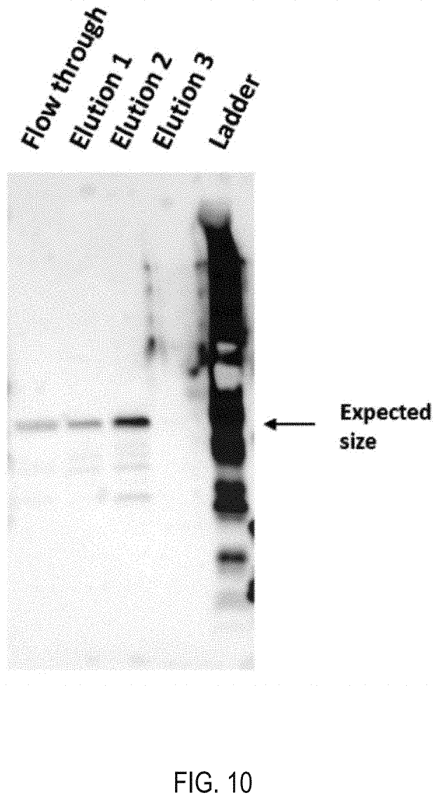

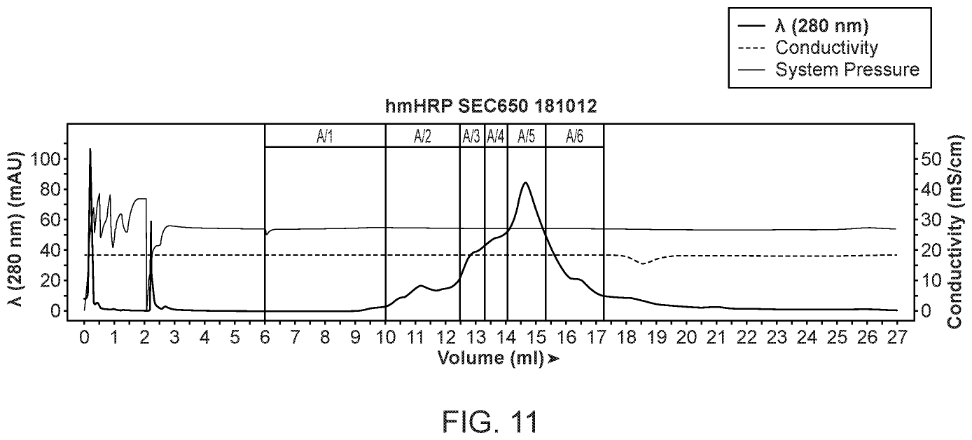

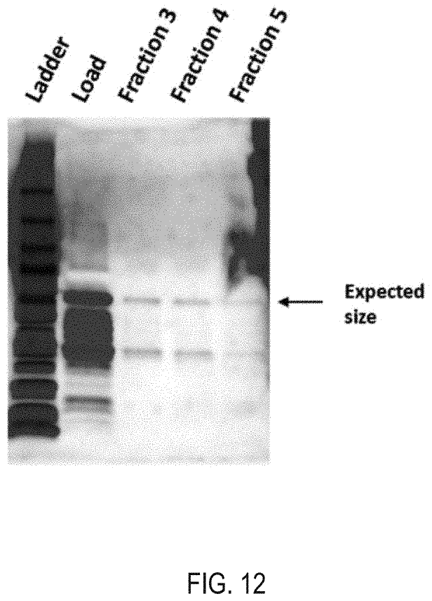

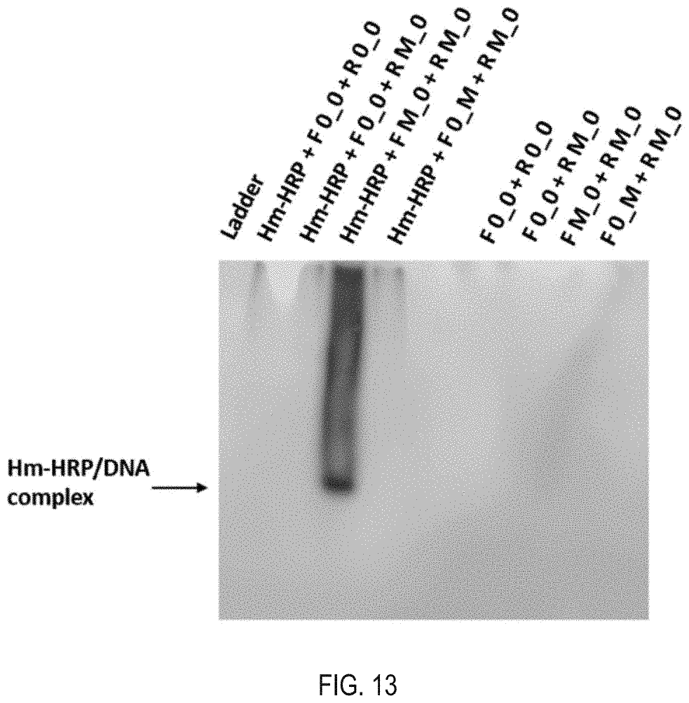

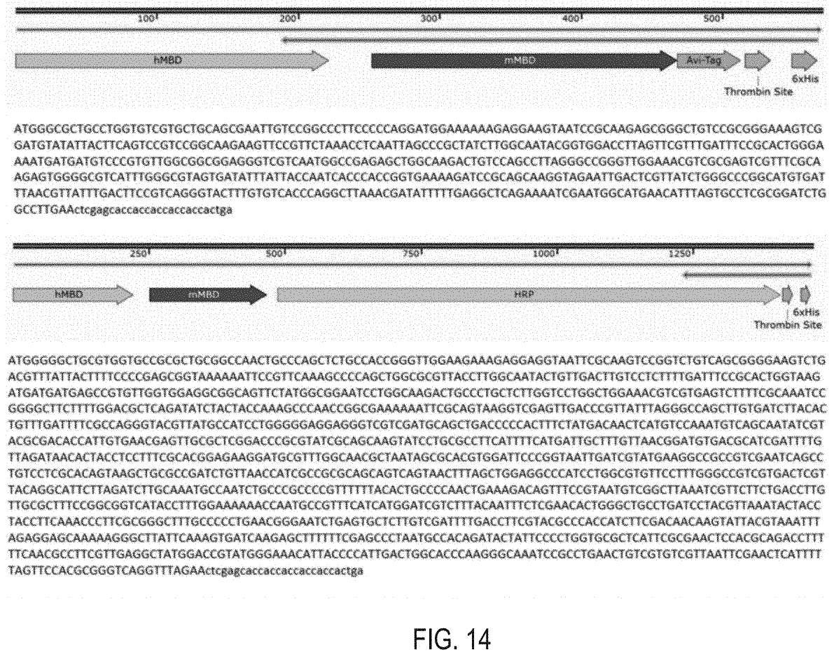

At step 210, the determined ctDNA concentration and corresponding methylation levels of the target ctDNA is monitored on a digital health platform. Additional results from a study conducted in accordance with the principles of the present invention are illustrated in FIGS. 5-14.

While various illustrative embodiments of the invention are described above, it will be apparent to one skilled in the art that various changes and modifications may be made therein without departing from the invention. The appended claims are intended to cover all such changes and modifications that fall within the true scope of the invention.

Throughout and within this disclosure reference is made to various technical and patent literature, the contents of which is hereby incorporated by reference in to the present disclosure to more fully describe the state of the art to which the disclosure pertains.

What is claimed is:

1. A method for performing a liquid biopsy, the method comprising:
contacting DNA probes with a sample of target ctDNA, the DNA probes comprising a graphene oxide (GO) interacting region and a target recognition region complementary to a target region of the target ctDNA based on biomarkers for identifying key CpG sites of the target ctDNA identified via a machine learning algorithm;
contacting the sample of target ctDNA with a labeled methyl-binding domain protein (MBD); and
determining ctDNA concentration and corresponding methylation levels of the target ctDNA based on fluorescence of the sample of target ctDNA; and
monitoring the determined ctDNA concentration and corresponding methylation levels of the target ctDNA on a digital health platform.

2. The method of claim 1, wherein the machine learning algorithm generates a model that uses clustering and logistic regression based on a training data set, and corroborates the model against a validation dataset to determine its diagnostic accuracy.

3. The method of claim 2, wherein the machine learning algorithm comprises a Random Forest algorithm.

4. The method of claim 2, wherein the machine learning algorithm comprises a LASSO regression algorithm.

5. The method of claim 1, wherein the one or more DNA probes further comprise a fluorescent dye.

6. The method of claim 1, wherein the one or more DNA probes comprise 10-150 base pairs.

7. The method of claim 1, wherein the GO interacting region of the one or more DNA probes comprises a high Guanine-Cytosine content.

8. The method of claim 1, wherein the MBD label comprises horse radish peroxidase (HRP) or green fluorescent protein (GFP).

9. The method of claim 1, further comprising pre-incubating the DNA probes with GO.

10. The method of claim 1, further comprising contacting the sample of target ctDNA with an Exonuclease III solution.

11. The method of claim 1, further comprising contacting the sample of target ctDNA with a hydrogen peroxide and 4-hydroxyphenylacetic acid solution.

12. The method of claim 1, wherein monitoring the determined ctDNA concentration and corresponding methylation levels of the target ctDNA on a digital health platform is used to determine whether an individual is predisposed to at least one of carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

13. The method of claim 1, further comprising assessing post-therapeutic effects of a medication by comparing the data indicative of the determined ctDNA concentration and corresponding methylation levels before and after a treatment using the medication.

14. A system for performing a liquid biopsy, the system comprising:
non-transitory computer readable media having instructions that, when executed by a processor cause the processor to execute a machine learning algorithm to identify one or more biomarkers for identifying key CpG sites of a target ctDNA;
a diagnostic cell-free protein-based system configured to determine ctDNA concentration and corresponding methylation levels of the target ctDNA based on measured fluorescence of a sample of target ctDNA in contact with DNA probes comprising a graphene oxide (GO) interacting region and a target recognition region complementary to a target region of the target ctDNA based on the identified biomarkers; and
a digital health platform configured to monitor the determined ctDNA concentration and corresponding methylation levels of the target ctDNA.


SEQUENCE LISTING (9 sequences)

SEQ ID NO: 1 - DNA, 24 bp, Artificial Sequence
  Description of Artificial Sequence Synthetic probe
atgcacgccgggattcctctgctg

SEQ ID NO: 2 - DNA, 24 bp, Artificial Sequence
  Description of Artificial Sequence Synthetic probe
cagcagaggaatcccggcgtgcat

SEQ ID NO: 3 - DNA, 24 bp, Artificial Sequence
  Description of Artificial Sequence Synthetic probe
  modified_base (6)..(6): iMe-dC
atgcacgccgggattcctctgctg

SEQ ID NO: 4 - DNA, 24 bp, Artificial Sequence
  Description of Artificial Sequence Synthetic probe
  modified_base (18)..(18): iMe-dC
cagcagaggaatcccggcgtgcat

SEQ ID NO: 5 - DNA, 24 bp, Artificial Sequence
  Description of Artificial Sequence Synthetic probe
  modified_base (9)..(9): iMe-dC
atgcacgccgggattcctctgctg

SEQ ID NO: 6 - DNA, 24 bp, Artificial Sequence
  Description of Artificial Sequence Synthetic probe
  modified_base (15)..(15): iMe-dC
cagcagaggaatcccggcgtgcat

SEQ ID NO: 7 - protein, 6 aa, Artificial Sequence
  Description of Artificial Sequence Synthetic 6xHis tag
HHHHHH

SEQ ID NO: 8 - DNA, 570 bp, Artificial Sequence
  Description of Artificial Sequence Synthetic polynucleotide
atgggcgctgcctggtgtcgtgctgcagcgaattgtccggcccttcccccaggatggaaaaaagaggaagtaatccgcaagagcgggctgtccgcgggaaagtcggatgtatattacttcagtccgtccggcaagaagttccgttctaaacctcaattagcccgctatcttggcaatacggtggaccttagttcgtttgatttccgcactgggaaaatgatgatgtcccgtgttggcggcggagggtcgtcaatggccgagagctggcaagactgtccagccttagggccgggttggaaacgtcgcgagtcgtttcgcaagagtggggcgtcatttgggcgtagtgatatttattaccaatcacccaccggtgaaaagatccgcagcaaggtagaattgactcgttatctgggcccggcatgtgatttaacgttatttgacttccgtcagggtactttgtgtcacccaggcttaaacgatatttttgaggctcagaaaatcgaatggcatgaacatttagtgcctcgcggatctggccttgaactcgagcaccaccaccaccaccactga

SEQ ID NO: 9 - DNA, 1470 bp, Artificial Sequence
  Description of Artificial Sequence Synthetic polynucleotide
atgggggctgcgtggtgccgcgctgcggccaactgcccagctctgccaccgggttggaagaaagaggaggtaattcgcaagtccggtctgtcagcggggaagtctgacgtttattacttttccccgagcggtaaaaaattccgttcaaagccccagctggcgcgttaccttggcaatactgttgacttgtcctcttttgatttccgcactggtaagatgatgatgagccgtgttggtggaggcggcagttctatggcggaatcctggcaagactgccctgctcttggtcctggctggaaacgtcgtgagtcttttcgcaaatccggggcttcttttggacgctcagatatctactaccaaagcccaaccggcgaaaaaattcgcagtaaggtcgagttgacccgttatttagggccagcttgtgatcttacactgtttgattttcgccagggtacgttatgccatcctgggggaggagggtcgtcgatgcagctgacccccactttctatgacaactcatgtccaaatgtcagcaatatcgtacgcgacaccattgtgaacgagttgcgctcggacccgcgtatcgcagcaagtatcctgcgccttcattttcatgattgctttgttaacggatgtgacgcatcgattttgttagataacactacctcctttcgcacggagaaggatgcgtttggcaacgctaatagcgcacgtggattcccggtaattgatcgtatgaaggccgccgtcgaatcagcctgtcctcgcacagtaagctgcgccgatctgttaaccatcgccgcgcagcagtcagtaactttagctggaggcccatcctggcgtgttcctttgggccgtcgtgactcgttacaggcattcttagatcttgcaaatgccaatctgcccgccccgttttttacactgccccaactgaaagacagtttccgtaatgtcggcttaaatcgttcttctgaccttgttgcgctttccggcggtcatacctttggaaaaaaccaatgccgtttcatcatggatcgtctttacaatttctcgaacactgggctgcctgatcctacgttaaatactacctaccttcaaacccttcgcgggctttgccccctgaacgggaatctgagtgctcttgtcgattttgaccttcgtacgcccaccatcttcgacaacaagtattacgtaaatttagaggagcaaaaagggcttattcaaagtgatcaagagcttttttcgagccctaatgccacagatactattcccctggtgcgctcattcgcgaactccacgcagacctttttcaacgccttcgttgaggctatggaccgtatgggaaacattaccccattgactggcacccaagggcaaatccgcctgaactgtcgtgtcgttaattcgaactcatttttagttccacgcgggtcaggtttagaactcgagcaccaccaccaccaccactga